(19) United States
(12) Patent Application Publication
(10) Pub. No.: US 2010/0216706 A1
(43) Pub. Date: Aug. 26, 2010

(54) Ghrelin Protects Substantia Nigra Dopamine Neurons

(76) Inventors: Tamas L. Horvath, New Haven, CT (US); Zane B. Andrews, New Haven, CT (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(21) Appl. No.: 12/600,122

(22) PCT Filed: May 13, 2008
(86) PCT No.: PCT/US08/06096

§ 371 Date: Nov. 13, 2009

Related U.S. Application Data
(60) Provisional application No. 60/930,345, filed on May 15, 2007.

(51) Int. Cl. A61K 38/16; A61K 31/438; A61K 31/4162; A61K 31/165; A61K 31/55; A61K 31/4178; A61K 38/07; A61P 25/00; C12N 5/0793

(52) U.S. Cl. 514/12; 514/278; 514/405; 514/616; 514/212.07; 514/397; 514/18; 435/375

(57) ABSTRACT

A method of treating neurodegeneration of substantia nigra pars compacta (SNpc) dopamine neurons and compositions therefor are provided.

### BACKGROUND OF THE INVENTION

Recent human studies have shown that body mass index, midlife adiposity and diabetes are associated with the neurodegenerative illness, Parkinson's Disease (PD) (Abbott et al., 2002, Neurology 59: 1051-7; Hu et al., 2007, Diabetes Care 30, 842-7; Hu et al., 2006, Neurology 67: 1955-9). Furthermore, obesity is a risk factor for chemically-induced neurodegeneration in mice (Choi et al., 2005, Free Radic Biol Med 38: 806-16). MPTP (1-methyl-4-phenyl-1,2,5,6 tetrahydropyridine) is a mitochondrial toxin that models PD by ablating nigral dopamine (DA) neurons. Specifically, MPTP causes dopamine cell death after biotransformation to MPP+ and entry into dopamine cells via the dopamine transporter (DAT) by inhibiting mitochondrial complex 1 activity, promoting reactive oxygen species (ROS) production, oxidative stress and caspase activation (Dauer et al., 2003, Neuron 39:889-909). Calorie restriction attenuates MPTP-induced neurotoxicity in non-human primates (Maswood et al., 2004, Proc Natl Acad Sci USA 101: 18171-6) and mice (Duan et al., 1999, J Neurosci Res 57: 195-206). Levels of the gut hormone, ghrelin, are inversely related to related to obesity, such that ghrelin levels are higher during negative energy balance or calorie restriction, and lower during positive energy balance or obesity (Tschop et al., 2001, Diabetes 50: 707-9). The only known ghrelin receptor, growth hormone secretagogue receptor 1a (GHSR), has been shown to be present in the substantia nigra pars compacta (SNpc) (Guan et al., 1997, Brain Res Mol Brain Res 48: 23-9). While the acute effect of ghrelin on dopamine neurons of the ventral tegmental area has been demonstrated (Abizaid et al., 2006, J Clin Invest. 116(12):3229-39. Epub 2006 Oct. 19), to date, there is no evidence that the peripheral metabolic hormone ghrelin plays a role in the function of SNpc DA neurons.

PD is a degenerative disorder of the central nervous system. PD is characterized by the progressive degeneration of dopamine (DA) neurons projecting from the SNpc to the dorsal striatum. The resulting loss of dopamine in the striatum leads to debilitating motor dysfunction including rigidity, resting tremor, postural instability and bradykinesia, the four primary symptoms of PD. Familial or genetic causes of PD only account for 10% of all cases, whereas 90% are considered sporadic and may manifest as a result of variety of environment factors.

There is no cure for PD. There are a number of medications that are effective for relieving some of the symptoms of PD. Levodopa, which nerve cells convert to dopamine, is effective for alleviating bradykinesia and rigidity in a large number of cases. However, levodopa is not particularly effective for alleviating tremor. Other PD drugs include dopamine agonists, which cause neurons to react as though sufficient dopamine were present. As PD progresses, however, the disease response to the medications currently available becomes less predictable. Furthermore, in some cases, the disease does not respond to currently-available drugs at all. Thus, there is a need in the art for additional therapeutics for Parkinson's disease. The present invention addresses this need.

### BRIEF SUMMARY OF THE INVENTION

The invention provides a method of treating neurodegeneration of a substantia nigra pars compacta (SNpc) dopamine neuron in a mammal. The method comprises administering a therapeutically effective amount of ghrelin or a ghrelin mimetic to a mammal diagnosed with SNpc neurodegeneration, wherein said ghrelin or ghrelin mimetic induces increased dopamine neuron function in said mammal, thereby treating SNpc neurodegeneration. Preferably, the mammal is human. Preferably, the SNpc neurodegeneration is associated with Parkinson's disease.

In an embodiment, increased dopamine neuron function comprises at least one of: increased firing rate of SNpc DA neurons, increased dopamine concentration in dorsal striatum, increased tyrosine hydroxylase mRNA, increased mitochondrial respiration and increased mitochondrial proliferation.

In an embodiment, ghrelin is administered. In another embodiment, a ghrelin mimetic is administered. In one aspect, the ghrelin mimetic is selected from the group consisting of: LY444711, MK-677, L-692,429, CP-424,391, NNC 26-0703, Growth hormone (GH) releasing hexapeptide (GHRP)-6, EP 1572 and Ape-Ser(Octyl)-Phe-Leu-aminoethylamide.

Administration may be selected from the group consisting of parenteral, oral, intranasal and recombinant.

The invention further provides a method of activating a dopamine neuron of the substantia nigra pars compacta (SNpc). The method comprises administering a therapeutically effective amount of ghrelin or a ghrelin mimetic to a SNpc dopamine (DA) neuron, wherein ghrelin or the ghrelin mimetic increases firing rate of the SNpc DA neuron.

In an embodiment, ghrelin is administered. In another embodiment, a ghrelin mimetic is administered. In one aspect, the ghrelin mimetic is selected from the group consisting of: LY444711, MK-677, L-692,429, CP-424,391, NNC 26-0703, Growth hormone (GH) releasing hexapeptide (GHRP)-6, EP 1572 and Ape-Ser(Octyl)-Phe-Leu-aminoethylamide.

In one embodiment, the SNpc DA neuron is in vitro.

In another embodiment, the neuron is in vivo. Administration in vivo includes parenteral, oral, intranasal and recombinant.

Additionally, a method of weight loss associated with Parkinson's disease in a human is provided. The method comprises administering a therapeutically effective amount of ghrelin or a ghrelin mimetic to the human having Parkinson's disease associated weight loss, wherein the ghrelin or ghrelin mimetic increases appetite in the human.

In an embodiment, ghrelin is administered. In another embodiment, a ghrelin mimetic is administered. In one aspect, the ghrelin mimetic is selected from the group consisting of: LY444711, MK-677, L-692,429, CP-424,391, NNC 26-0703, Growth hormone (GH) releasing hexapeptide (GHRP)-6, EP 1572 and Ape-Ser(Octyl)-Phe-Leu-aminoethylamide.

Administration may be selected from the group consisting of parenteral, oral, intranasal and recombinant.

A method of assessing if a mammal is at risk of developing SNpc neurodegeneration is also provided. The method comprises assessing endogenous production and/or secretion of ghrelin in a mammal. If the production and/or secretion of ghrelin are reduced compared to a reference level designated as normal, the mammal is at risk of developing SNpc neurodegeneration. Preferably, the mammal is a human. Assessing endogenous production and/or secretion of ghrelin may comprise an immunoassay or a gene expression assay.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-C are a series of images related to ghrelin binding in the substantia pars compacta (SNpc). FIGS. 1A1 and 1A2 are low and high power fluorescent images, respectively, of biotinylated ghrelin binding in the SNpc. Arrows in A1 depicts neurons enlarged in A2. FIG. 1A3 is a fluorescent image of SNpc with unbiotinylated ghrelin. FIG. 1B is an image of growth hormone secretagogue receptor 1a (GHSR) immunostaining and depicts the typical angular orientation of SNpc dopamine neurons. FIG. 1C1 is a high power fluorescent image of dual channel GHSR and tyrosine hydroxylase (TH) immunoreactivity in the SNpc. The solid white arrows depict representative GHSR immunoreactivity. The dotted white arrow points to representative regions of TH immunoreactivity. FIG. 1C2 is a single channel fluorescent image of the GHSR immunoreactivity shown in C1. Scale bar: in A1=100 microns; in A2, A3, C2=20 microns; in B=30 microns.

FIGS. 2A-2D depict data related to ghrelin regulation of nigrostriatal dopamine (DA) function. FIG. 2A is a bar graph of SNpc DA action potential frequency under different conditions. (p<0.05; *, significantly increased compared to controls). FIG. 2B depicts representative action potential trace recordings from control, ghrelin and washout conditions. FIG. 2C is a bar graph of DA concentration (ng/mg protein) in the dorsal striatum (n=7 per group, p<0.05; *, significantly increased compared to controls). FIG. 2D is a bar graph of the fold increase in expression of TH mRNA in the midbrain (n=8 per group, p=0.0013; *, Significantly increased compared to controls).

FIGS. 3A-3E are a series of bar graphs related to the effect of ghrelin on locomotor activity of ghrelin knockout (ghrelin ko) mice compared to wild type (wt) mice in an open field activity test. FIG. 3A is a bar graph of total movement time for wt and ghrelin ko mice. (*p<0.01) FIG. 3B is a bar graph total movement distance. (*p<0.001). FIG. 3C is a bar graph of distance per move (*p<0.001). FIG. 3D is a bar graph of mean velocity (*p<0.001). FIG. 3E is a bar graph of rest time (*p<0.01). ghr^{-/-}=ghrelin knockout. Wt mice (n=9). Ghrelin ko mice (n=8).

FIGS. 4A-4D are a series of graphs and images relating to ghrelin and mitochondrial respiration and proliferation in the substantia nigra. FIG. 4A is a bar graph of mitochondrial respiration in the midbrain of ghrelin-treated rats in the presence of different compounds. (*Significant with respect saline rats (n=5 per group). PYR=pyruvate. Mal=malate. FCCP=carbonyl cyanide p-(trifluoromethoxy)phenylhydrazone.) FIGS. 4B and 4C are images of SNpc DA neurons after saline injection (4B) or ghrelin injection (4C). Asterisks indicate mitochondria. Scale bar=1 micrometer (μm). FIG. 4D is a bar graph of mitochondria number in DA neurons from saline- or ghrelin-injected mice. (*Significant respect to wt saline, n=13-15).

FIGS. 5A-5I are a series of images and graphs relating to the effect of ghrelin on MPTP-induced dopamine cell loss in the SNpc. FIGS. 5A-5C depict representative images of TH cell number in SNpc of mice treated with saline for 14 days with saline on day 7 (5A), saline for 14 days with MPTP on day 7 (5B), or ghrelin for 14 days with MPTP on day 7 (5C). “ML” refers to the medial lemniscus, which served as a reference point to identify sections at equal positions relative to bregma. “SNr” refers to substantia nigra pars reticulata. FIG. 5D is an image from the mouse brain atlas. Note the level of the SNpc at −3.08 mm bregma. The X-axis of FIGS. 5E through 5I is the treatment agent injected on day 7 (saline or MPTP) of a 14 day treatment paradigm with saline (white bars) or ghrelin (black bars). FIG. 5E is a graph depicting the stereological quantification of total TH cell number in the SNpc. FIG. 5F is a graph depicting SNpc volume data. FIG. 5G depicts MPTP-induced striatal dopamine loss as a function of saline treatment vs ghrelin treatment. FIGS. 5H and 5I depict striatal DOPAC (metabolite of dopamine) concentration and the DOPAC/dopamine ratio, as an index of dopaminergic activity at the nerve terminal in the striatum. (a, Significant with respect to non-MPTP injected saline controls. b, significant with respect to ghrelin/MPTP treated mice. Data analyzed using ANOVA followed by bonferroni post hoc test, p<0.05, n=6 in all groups).

FIGS. 6A-6D are a series of graphs relating to ghrelin effect on uncoupling protein 2 (UCP2)-dependent mitochondrial respiration and proliferation. FIG. 6A is a bar graph of mitochondrial respiration after addition of oligomycin. Mice received intraperitoneal (IP) ghrelin injection 3 hours earlier (30 nmol). FIG. 6B is a bar graph of uncoupling activity after ghrelin treatment and the addition of palmitate. FIG. 6C is a bar graph of total uncoupling activity after ghrelin injection and FCCP. (*Significant with respect to wt saline. n=4. Each sample represents 3 pooled midbrain dissections). FIG. 6D is a bar graph of mitochondrial number in SNpc dopamine neurons after ghrelin injection two hours earlier. (*Significant respect to wt saline. n=13-15).

FIGS. 7A-7E are a series of images and graphs related to susceptibility to SNpc DA cell loss after MPTP intoxication in mice lacking the ghrelin gene relative to wild type controls. FIGS. 7A-7C are representative lower power images showing DA cell number in SNpc after saline (7A; n=10), MPTP to ghrelin wt (ghrelin^{+/+}) mice (7B; n=8) or MPTP to ghrelin ko (ghrelin^{-/-}) mice (7C; n=9). FIG. 7D is a graph depicting the quantification of total TH cell number in ghrelin wt and ghrelin ko mice treated with either saline or MPTP. Quantification was conducted using the optical fractionator by unbiased stereology. FIG. 7E is a graph of striatal dopamine quantity in ghrelin^{+/+} and ghrelin^{-/-} mice treated with either saline or MPTP. (*Significant with respect to saline controls; # significant with respect to wt MPTP).

FIGS. 8A-8E are a series of images and graphs related to MPTP-induced DA cell loss after selective reactivation of GHSR in TH neurons. FIGS. 8A-8C are representative dark-field photomicrographs of in situ hybridization histochemistry (ISHH) experiments performed on mouse brains using a mouse GHSR-specific riboprobe. GHSR mRNA expression is evidenced by the white-appearing silver granules. SN=substantia nigra. VTA=ventral tegmental area. FIG. 8A is a representative image of wild type mice, showing normal level of GHSR mRNA expression in SN and VTA. FIG. 8B is a representative image of GHSR-null mice, depicting the lack of GHSR transcripts in SN and VTA. FIG. 8C is a representative image of “TH-only” mice with re-activated GHSR mRNA expression in SN and VTA. FIGS. 8D-8G are representative low power images of TH staining in the SNpc after MPTP insult (n=6 all groups) in all genotypes (8E: wt/wt; 8F: homo/wt; 8G: homo/TG), compared to wt/wt saline (8D). FIG. 8H is a graphical representation of TH cell number in the SNpc, as quantified by stereology using the optical fractionator. (*, Significant with respect to saline-treated mice. #, Significant with respect to homo/wt MPTP. Data analyzed using ANOVA followed by bonferroni post hoc test, p<0.05, n=6 in all groups).

### DETAILED DESCRIPTION OF THE INVENTION

The present invention springs in part from the discovery that exogenously administered ghrelin plays a role in the maintenance and protection of normal nigrostriatal dopamine function. Specifically, ghrelin activates substantia nigra pars compacta dopamine neurons, increasing dopamine levels and tyrosine hydroxylase mRNA levels. In addition, ghrelin protects SNpc DA cells from toxic insult. Ghrelin also increases mitochondrial respiration and number in SNpc.

Accordingly, the invention provides methods of treating neurodegeneration of substantia nigra pars compacta (SNpc) dopamine neurons and compositions therefor. In one embodiment, a method of treating Parkinson's disease by administering ghrelin or a ghrelin mimetic is provided.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

The techniques and procedures for recombinant manipulations, including nucleic acid and peptide synthesis, are generally performed according to conventional methods in the art and various general references (e.g., Sambrook et al, 2001, Molecular Cloning, A Laboratory Approach, Cold Spring Harbor Laboratory Press, Cold Spring Harbor, N.Y.; Ausubel et al., eds, 2005, Current Protocols in Molecular Biology, John Wiley & Sons, New York, N.Y.; and Gerhardt et al., eds., 1994, Methods for General and Molecular Bacteriology, American Society for Microbiology, Washington, D.C.), which are provided throughout this document.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used. As used herein, a “ghrelin mimetic” refers to a molecule which induces at least the biological activity of ghrelin of binding to a substantia nigra pars compacta ghrelin receptor and promoting at least one of: increase the firing rate of SNpc DA neurons, increase dopamine concentration in dorsal and/or ventral striata, increase tyrosine hydroxylase (TH) mRNA, alleviate or eliminate motor dysfunction, increase mitochondrial respiration in SNpc and increase mitochondrial proliferation in SNpc. Other biological activities of ghrelin include: calcium mobilization, inositol phosphate turnover, c-AMP-response element or serum-response element controlled transcription, and arrestin mobilization (see Hoist et al, 2005, Mol. Endocrinol. 19(9):2400-2411).

As used herein, a “ghrelin receptor” refers to any naturally-occurring molecule to which ghrelin binds and induces a biological activity. Ghrelin is known to bind to growth hormone secretagogue receptor 1a (GHSR); however, the invention should not be construed as limited to a specific type of receptor.

As used herein, an “agonist” is a composition of matter which, when administered to a mammal such as a human, enhances or extends a biological activity attributable to the level or presence of an endogenous biologically active molecule in the mammal.

“Treating,” as used herein, means ameliorating the effects of, or delaying, halting or reversing the progress of a condition. The word encompasses reducing the severity of a symptom of a condition and/or the frequency of a symptom of a medical condition.

A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein, “therapeutically effective amount” refers to a nontoxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

“Pharmaceutically acceptable carrier” refers herein to a composition suitable for delivering the inventive composition to a subject without excessive toxicity or other complications while maintaining the biological activity of the molecule. Protein-stabilizing excipients, such as mannitol, sucrose, polysorbate-80 and phosphate buffers, are typically found in such carriers, although the carriers should not be construed as being limited only to these compounds.

As used herein, the term “physiologically acceptable” ester or salt means an ester or salt form of an active ingredient in a pharmaceutical composition which is compatible with any other ingredients of the pharmaceutical composition and which is not deleterious to the subject to which the composition is to be administered.

As used herein “endogenous” refers to any material produced within or originating inside an organism.

“Exogenous” refers to any material introduced into or produced outside an organism.

As used herein, “expression cassette” refers to a nucleic acid molecule comprising a coding sequence operably linked to promoter/regulatory sequences necessary for transcription and translation of the coding sequence. An expression cassette encoding a desired nucleic acid sequence does not include translation sequences.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGCS′ share 50% homology.

As used herein, “homology” is used synonymously with “identity.” It is understood that any and all whole or partial integers between any ranges set forth herein are included herein.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is naturally occurring.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, a “peptidomimetic” is a compound containing non-peptidic structural elements that is capable of mimicking the biological action of a parent peptide. A peptidomimetic may or may not comprise peptide bonds.

“Polypeptide” refers to a polymer composed of amino acid residues, related naturally-occurring, structural variants, and synthetic, non-naturally-occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

“Specifically bind” as used herein refers to the higher affinity of a binding molecule for a target molecule compared to the binding molecule's affinity for non-target molecules. A binding molecule that specifically binds a target molecule does not substantially recognize or bind non-target molecules.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter” is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A “recombinant polypeptide” is one which is produced upon expression of a recombinant polynucleotide.

By the term “applicator,” as the term is used herein, is meant any device including, but not limited to, a hypodermic syringe, a pipette, and the like, for administering the compounds and compositions of the invention.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, “activating a neuron” refers to increasing the action potential frequency (i.e., firing rate) above baseline. Baseline frequency is the frequency in the absence of a ghrelin or ghrelin mimetic. A dopamine neuron releases dopamine when it is activated.

As used herein, an “immunoassay” refers to any binding assay that uses an antibody capable of binding specifically to a target molecule to detect and quantify the target molecule.

It is understood that any and all whole or partial integers between any ranges set forth herein are included herein.

### DESCRIPTION

As demonstrated herein, the administration of ghrelin or a ghrelin mimetic has an acute effect on the firing rate of substantia nigra pars compacta (SNpc) dopamine (DA) neurons. Ghrelin binding to growth hormone secretagogue receptor 1a (GHSR) in SNpc cells leads to increased tyrosine hydroxylase mRNA and increases dopamine concentration in the dorsal striatum. Furthermore, ghrelin enhances mitochondrial respiration and proliferation in SNpc DA neurons. While not wishing to be bound by theory, it is believed that ghrelin's effect on the firing rate of SNpc DA neurons enhances the functioning of surviving DA neurons during the course of degeneration, leading to a lower loss of dopamine levels in the dorsal striatum. Further, it is believed that ghrelin-induced enhancement of mitochondrial respiration and proliferation provides a better bioenergetic status that makes these neurons more resistant to cellular stress. While not being bound by theory, it is thought that this aspect of ghrelin's neuroprotective effect may be related to activation of uncoupling protein 2 (UCP2)-dependent respiration, an endogenous antioxidant mechanism.

Thus, the present invention relates to methods of treating neurodegeneration in substantia nigra pars compacta (SNpc) by administering ghrelin or a ghrelin mimetic to a patient in need thereof. Specifically, the methods of the invention are useful in treating any disease or disorder featuring neurodegeneration in SNpc dopamine neurons. Preferably, the methods are used to treat Parkinson's disease.

The methods are also useful for reducing weight loss in Parkinson's disease patients. The invention further disclosed herein provides a method of activating a SNpc dopamine neuron. A method of assessing if an individual is at risk of developing SNpc neurodegeneration is also provided.

Some examples of diseases which may be treated according to the methods of the invention are discussed herein. The invention should not be construed as being limited solely to these diseases, as other diseases featuring SNpc dopamine cell neurodegeneration disease pathology, which are at present unknown, once known, may also be treatable using the methods of the invention.

The therapeutic methods and compositions of the invention may be used in any animal subject, preferably a mammal. The mammal is preferably a veterinary animal, including a primate, and more preferably, the mammal is a human. The methods may also be used in many other mammalian subjects, including but not limited to mice, rats, dogs, cats, livestock and horses for veterinary purposes, and for drug screening and drug development purposes.

I. Ghrelin and Ghrelin Mimetics

The methods of the invention comprise administration of ghrelin or a ghrelin mimetic. Ghrelin is the endogenous ligand of GHSR. Naturally-occurring ghrelin is expressed as a 117 amino acid preprohormone sequence comprising a signal peptide followed by the ghrelin sequence, which is followed by a cleavage sequence as well as additional sequences, including a second hormone, obestatin. The naturally-occurring mature ghrelin peptide is a 28 amino acid acyl-peptide esterified with octanoic acid on Ser3. Acylation is required for ghrelin's action at GHSR.

The amino acid sequence for mature human ghrelin is Gly-Ser-Ser-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (SEQ ID NO. 1). An exemplary sequence encoding human ghrelin is

(SEQ ID NO. 2)

5′-ggctccagct tcctgagccc tgaacaccag agagtccagc
agagaaagga gtcgaagaag ccaccagcca agctgcagcc
ccga-3′.

The ghrelin gene has been cloned in numerous species, including monkey, mouse, rat, gerbil, pig, cat, dog, goat, sheep and cattle. The mature ghrelin sequence is highly conserved across species; the first 10 amino acids of the ghrelin gene from these eleven species are identical.

Any naturally-occurring ghrelin may be used in the methods of the invention. Preferably, the human ghrelin (SEQ ID NO. 1) is used. In addition, ghrelin mimetics, such as analogs and derivatives of a naturally-occurring ghrelin, are useful in the invention. Analogs can differ from naturally-occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Maximum ghrelin activity requires acylation of the third residue of ghrelin. Naturally-occurring ghrelin is acylated with octanoic acid, however, any bulky hydrophobic group attached to the side chain of the third residue is sufficient for ghrelin function (Matsumoto et al., 2001, Biochem. Biophys. Res. Commun. 287:142-146). Non-limiting examples of such hydrophobic groups include n-lauroyl, palmitoyl, 3-octenoyl and 4-methylpentanoyl. Furthermore, ghrelin derivatives wherein the ester bond between octanoic acid and Ser3 is more chemically stable, such as a thioether (Cys3(octyl)) or ether (Ser3(octyl) bond), are also useful in the practice of the present invention.

Ghrelin derivatives are also useful in the invention. Derivatives include truncation mutants of ghrelin. There is a wealth of information regarding the structure and function of ghrelin to guide the skilled artisan in preparing ghrelin derivatives useful in the present invention. See, for instance, Kojima et al., 2005, Physiol. Rev. 85: 495-522 and references cited therein. Structurally, ghrelin is a random coil in aqueous solution (Silva Elipe et al., 2001, Biopolymers 59:489-501). Various truncated ghrelin peptides also demonstrate random coil structure. The minimum active ghrelin core is the first four amino acids with Ser3 acylated (Bednarek et al., 2000, J. Med. Chem. 43:4370-4376; Matsumoto et al., 2001, Biochem. Biophys. Res. Commun. 284:655-659). Thus a ghrelin derivative comprising only the first four amino acids, e.g., Gly-Ser-Ser(n-octanoyl)-Phe (SEQ ID No. 3), is also useful in the present invention. Another ghrelin derivative useful in the practice of the invention is 5-aminopentanoyl-Ser(Octyl)-Phe-Leu-aminoethylamide (Ser-Phe-Leu are residues 3-5 of SEQ ID NO. 1), which was found to have potent ghrelin activity (Matsumoto et al., 2001, Biochem. Biophys. Res. Commun. 284:655-659).

Also included are ghrelin polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally-occurring L-amino acids, e.g., D-amino acids or non-naturally occurring synthetic amino acids. The peptides of the invention are not limited to products of any of the specific exemplary processes listed herein. The ghrelin polypeptides useful in the invention may further be conjugated to non-amino acid moieties that are useful in their therapeutic application. In particular, moieties that improve the stability, biological half-life, water solubility, and immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.), Handbook of Water Soluble Gums and Resins, McGraw-Hill, New York, chapter 18). PEG possesses an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

It will be appreciated, of course, that the peptides may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

In addition to analogs and derivatives of ghrelin, other types of ghrelin mimetics useful in practicing the invention include: peptidomimetics, small molecule mimetics and GHS-R agonists. A substantial number of ghrelin mimetics are known in the art. Non-limiting examples of ghrelin mimetics include LY444711, hexarelin, growth hormone releasing hexapeptide-1 (GHRP-1), GHRP-2, GHRP-6, ipamorelin, MK-0677, NN₇O₃, capromorelin, G7039, G7134, G7203, G7502, SM-130686, RC-1291, L-692429, L-692587, L-739943, L-163255, L-163540, L-163833, L-166446, CP-424391, EP-51389, NNC-26-0235, NNC-26-0323, NNC-26-0610, NNC 26-0703, NNC-26-0722, NNC-26-1089, NNC-26-1136, NNC-26-1137, NNC-26-1187, NNC-26-1291 and macrocyclic compounds (U.S. Publication No. 20060025566). See also Smith, 2005, Endo. Rev. 26:346-360 for information on developing ghrelin mimetics.

Polypeptides and nucleic acids useful in the methods of invention may be obtained by standard methods known to the skilled artisan. Methods include in vitro peptide synthesis and biological means. Biological means includes purification from a biological source, in vitro translation synthesis and recombinant synthesis using a recombinant host cell.

Merrifield-type solid phase peptide synthesis may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-60). A great advantage to the utilization of a synthetic peptide route is the ability to produce large amounts of peptides, even those that rarely occur naturally, with relatively high purities, i.e., purities sufficient for research, diagnostic or therapeutic purposes.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Biological preparation of ghrelin or ghrelin mimetic may include purification from a cell, tissue or organism that comprises the desired component. For instance, a naturally-occurring source of ghrelin is stomach extracts (Kojima et al., 1999, Nature 403:656-660). Biological preparation also includes expression of a gene or coding sequence for a ghrelin polypeptide in a recombinant host cell or an in vitro translation system. The DNA coding sequence for human ghrelin is provided in SEQ ID NO. 2. DNA and amino acid sequences are available for numerous other naturally-occurring ghrelin homologs and are readily obtained by the skilled artisan from publicly-available databases, such as GenBank® (United States Department of Health and Human Services, Bethesda Md.). The skilled artisan can readily design and obtain coding sequences for various ghrelin derivatives using standard molecular biology techniques.

Vectors for expression cassettes and methods for the introduction of exogenous DNA into cells with concomitant expression of the exogenous DNA in the cells are described, for example, in Sambrook et al., supra, 2001; Ausubel et al., supra, 2005. Techniques for introducing vectors into target cells include, but are not limited to, electroporation, photoporation, calcium precipitation, fusion, transfection, lipofection, viral targeting and the like.

Any expression vector compatible with the expression of a polypeptide in a host cell is suitable for use in the instant invention, and can be selected from the group consisting of a plasmid DNA, a viral vector, and a mammalian vector. Vectors may be episomal, or may be provided for integration into the target cell genome via homologous recombination or random integration. Viral vectors useful in the methods of the invention include, but are not limited to, cytomegalovirus vectors, adenovirus vectors and retrovirus vectors, such as MigRI, MMLC, HIV-2 and ALV.

The vector comprising the expression cassette, or a vector that is co-introduced with the expression vector, can comprise a marker gene. Marker genes are useful, for instance, to monitor transfection efficiencies. Marker genes include genes for selectable markers, including, but not limited to, G418, hygromycin, and methotrexate, and genes for detectable markers, including, but not limited to, luciferase and GFP.

The coding sequence contained in an expression cassette may, optionally, be fused in-frame to other coding sequences. For instance, the coding sequence of a detectable tag or purification tag may be included. Such tags are useful, for instance, to assist in the rapid purification of the encoded polypeptide or variant thereof. An example of such a tag is a 6-His sequence. The fusion may be at either the N-terminal or the C-terminal of a polypeptide, provided the biological activity of the molecule is maintained. Such tags may be removed from the purified fusion polypeptide by engineering an intervening cleavage site between the tag and the other coding sequence. Thrombin is a useful cleavage agent for this purpose. Commercial products, such as TAGZyme (Qiagen® Inc., Valencia, Calif.), are available as well.

In the context of an expression vector, the vector may be readily introduced into a suitable host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., supra, 2001 and Ausubel et al., supra, 2005.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems are well known in the art.

To ensure that the polypeptide obtained from either chemical or biological synthetic techniques is the desired polypeptide, analysis of the polypeptide composition may be conducted. Such amino acid composition analysis may be conducted using high resolution mass spectrometry to determine the molecular weight of the peptide. Alternatively, or additionally, the amino acid content of the peptide may be confirmed by hydrolyzing the peptide in aqueous acid, and separating, identifying and quantifying the components of the mixture using HPLC or an amino acid analyzer. Protein sequenators, which sequentially degrade the peptide and identify the amino acids in order, may also be used to definitively determine the sequence of the peptide. One of skill in the art is familiar with conventional methods for analyzing a nucleic acid antigen and other types of antigens.

Prior to use in the compositions and methods of the invention, polypeptides or other molecules are optionally purified to remove contaminants. Any one of a number of a conventional purification procedures may be used to attain the required level of purity including, for example, reversed-phase high-pressure liquid chromatography (HPLC) using an alkylated silica column, such as C₄-, C₈- or C₁₈-silica, or variations thereof. A gradient mobile phase of increasing organic content is generally used to achieve purification, for example, acetonitrile in an aqueous buffer, usually containing a small amount of trifluoroacetic acid. Ion-exchange chromatography may be also used to separate polypeptides based on their charge. Gel filtration chromatography may be used to separate polypeptides, nucleic acids and other types of antigens based on their size.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

Ghrelin mimetics that are not nucleic acids or polypeptides may also be obtained by conventional methods known in the art. Such mimetics may be purified from a naturally-occurring source or synthesized biologically (e.g., enzymatic production in vitro or using recombinantly-engineered organisms) or chemically (e.g., organic synthesis). Purification methods include, but are not limited to, ultrafiltration, nanofiltration, reverse osmosis, high pressure liquid chromatography, and the like. Methods used in analytical chemistry and organic syntheses are well known and commonly employed in the art. Standard techniques or modifications thereof, may be used for chemical syntheses and chemical analyses.

II. Administration of Pharmaceutical Compositions

The therapeutic methods of the invention encompass the use of pharmaceutical compositions comprising ghrelin or a ghrelin mimetic for administration in accordance with the present invention. The pharmaceutical compositions useful for practicing the invention may be administered to deliver a dose of between about 1 ng/kg/day and about 100 mg/kg/day, and any and all whole or partial increments therebetween. In one embodiment, the invention envisions administration of a dose which results in a concentration of the compound of the present invention between about 1 μM and about 10 μM in a mammal.

Typically, dosages of ghrelin or a ghrelin mimetic which may be administered to an animal, preferably a human, range in amount from about 1 μg to about 100 g per kilogram of body weight of the animal, and any and all whole or partial increments therebetween. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The pharmaceutical composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Any route of administration is suitable for use in the therapeutic methods of the invention. Examples of routes of administration include oral, rectal, vaginal, parenteral, topical, pulmonary, intranasal, buccal, ophthalmic, intrathecal or another route of administration. Recombinant administration, such as administration of DNA encoding ghrelin or a ghrelin mimetic, is also contemplated. Accordingly, pharmaceutical compositions that are useful in the methods of the invention may be prepared, packaged, or sold in formulations suitable for oral, rectal, vaginal, parenteral, topical, pulmonary, intranasal, intravenous, epidural, intraspinal, intra-arterial, buccal, ophthalmic, intrathecal, recombinant or another route of administration. Other contemplated formulations include projected nanoparticles, liposomal preparations, resealed erythrocytes containing the active ingredient, and immunologically-based formulations.

Preferred routes of administration are intravenous, intranasal, oral, intraperitoneal and subcutaneous.

The invention encompasses the preparation and use of pharmaceutical compositions comprising ghrelin or a ghrelin mimetic as an active ingredient that are useful for treatment of the diseases disclosed herein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Such active agents include, but are not limited to, levadopa, carbidopa and dopamine agonists, such as bromocriptine, apomorphine, pramipexole and ropinirole. In some instances, additional agents include anti-obesity agents and/or appetite suppressants.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, intracerebroventricular, surgical implant, internal surgical paint and kidney dialytic infusion techniques.

Preferred parenteral administrations are intravenous, intraperitoneal and subcutaneous.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Ghrelin or a ghrelin mimetic may also be administered recombinantly. Recombinant administration of ghrelin or a ghrelin mimetic is accomplished by methods known in the art, such delivery of a nucleic acid comprising an expression cassette encoding ghrelin or a mimetic thereof to the target cell. Within the expression cassette, the coding polynucleotide is operably linked to a suitable promoter. Examples of suitable promoters include prokaryotic promoters and viral promoters (e.g., retroviral ITRs, LTRs, immediate early viral promoters (IEp), such as herpesvirus IEp (e.g., ICP4—IEp and ICP0-IEEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters, such as enhancers (e.g., the rabbit β-globin regulatory elements), constitutively active promoters (e.g., the β-actin promoter, etc.), signal specific promoters (e.g., inducible promoters such as a promoter responsive to RU486, etc.), and tissue-specific promoters. It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the coding sequence for ghrelin or a ghrelin mimetic should be incorporated into a genetic vector suitable for delivering the coding sequence to the target cells. Cells may be targeted in vivo in the subject or targeted ex vivo, after which the modified cells are administered to the subject. Suitable cells for in vivo or ex vivo targeting are dopamine cells in the substantia nigra pars compacta. Precursor dopamine cells, such as neural stem or progenitor cells, are also contemplated for use in delivering the coding sequence to the target cells. Depending on the desired end application, any genetic vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). The choice of vector will largely determine the method used to introduce the vector into the cells (e.g., by protoplast fusion, calcium-phosphate precipitation, gene gun, electroporation, DEAE dextran or lipid carrier mediated transfection, infection with viral vectors, etc.), which are generally known in the art.

III. Other Methods

As shown herein, ghrelin ko mice are more susceptible to DA cell loss in the SNpc and dopamine loss in the striatum after MPTP. Thus, it is believed that any factor leading to reduced ghrelin production or secretion, genetic or environmental, could predispose individuals to nigrostriatal dopaminergic dysfunction. Accordingly, a method of assessing if an individual is at risk of developing SNpc neurodegeneration is provided. The method comprises assessing endogenous production level and/or secretion level of ghrelin in an individual or a biological sample obtained therefrom, wherein if the production level and/or secretion level of ghrelin is reduced compared to a reference level, the individual is said to be at higher risk of developing SNpc neurodegeneration compared to the normal risk. As used herein, a “reference level” refers to a level, or range of levels, of ghrelin production or secretion obtained from one mammal or more than one mammal designated as normal, that is, without clinical manifestation of SNpc neurodegeneration. As used herein, a level is “reduced compared to a reference level” if the level is smaller than the reference level, and the difference between the levels is statistically significant. The skilled artisan is familiar with methods in the art for determining such a reference level and establishing statistical significance. Any biological sample containing ghrelin in vivo may be used in the method. In one embodiment, the biological sample is plasma.

Assessing ghrelin production and/or secretion may be performed using any one of a number of known methods. In an embodiment, the level of ghrelin in a biological sample is assessed using high pressure liquid chromatography. In another embodiment, the level of ghrelin in a biological sample is assessed using an immunoassay.

Immunoassays useful in the present invention include, for example, immunohistochemistry assays, immunocytochemistry assays, ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor Laboratory Press, Cold Spring Harbor, N.Y.; Harlow et al., 1999, Using Antibodies: A Laboratory Manual, Cold Spring Harbor Laboratory Press, Cold Spring Harbor, N.Y. The preparation of polyclonal and monoclonal antibodies is well known in the art. In addition, antibodies to ghrelin are commercially available.

Ghrelin production may also be assessed by quantitating ghrelin transcripts using standard gene expressions methods, such as real-time-PCR.

Parkinson's disease is associated with a decrease in appetite, leading to weight loss. Ghrelin is an appetite stimulating hormone. Administration of ghrelin or a ghrelin mimetic is therefore expected to alleviate or limit weight loss in Parkinson's disease patients.

The invention also encompasses a method of activating a dopamine neuron of the substantia nigra pars compacta (SNpc). The method comprises the step of administering a therapeutically effective amount of ghrelin or a ghrelin mimetic to a SNpc dopamine (DA) neuron, wherein the ghrelin or ghrelin mimetic increases firing rate of the SNpc DA neuron. The SNpc DA neuron may be in in vitro culture or may be in vivo.

The invention also encompasses the use of ghrelin or a ghrelin mimetic for the preparation of a medicament for the treatment of neurodegeneration of substantia nigra pars compacta (SNpc) dopamine (DA) neurons.

IV. Kits

The invention further provides a kit useful in the practice of the methods of the invention. In one embodiment, a kit comprising ghrelin or a ghrelin mimetic and an instructional material describing how to use ghrelin or the mimetic to treat SNpc neurodegeneration disease or disorder is provided. Optionally, the kit comprises an applicator for administration of ghrelin or a ghrelin mimetic. In an embodiment, the kit comprises a mammalian cell that recombinantly expresses ghrelin or a mimetic thereof.

In another embodiment, a kit useful for assessing if a mammal is at risk of developing SNpc neurodegeneration. The kit comprises an antibody to ghrelin and an instructional material describing how to use the antibody to assess risk. Optionally, the kit comprises a container for a biological sample. Optionally, the kit comprises a positive control and a negative control.

V. Identification of Ghrelin Mimetics

The instant invention also features methods for identifying ghrelin mimetics useful for treating SNpc neurodegeneration by screening test molecules for the ability to induce at least one of: increase the firing rate of SNpc DA neurons, increase dopamine concentration in dorsal and/or ventral striata, increase tyrosine hydroxylase (TH) mRNA, alleviate or correct motor dysfunction, increase mitochondrial respiration in SNpc, and increase mitochondrial proliferation in SNpc. “Increase” is relative term and is relative to the property observed in the absence of the test molecule. A test molecule that has at least one of these properties is identified as a therapeutic candidate for treating SNpc neurodegeneration. Preferably, a test molecule identified as a therapeutic candidate will have two or more of these properties. Most preferably, the test molecule will have all of these properties. Screening a test molecule may also comprise comparison to the effect of exogenously provided ghrelin; when that is the case, a test compound that induces at least one of: firing rate of SNpc DA neurons, dopamine concentration in dorsal and/or ventral striata, tyrosine hydroxylase (TH) mRNA, motor function, mitochondrial respiration in SNpc, and mitochondrial proliferation in SNpc, at a level comparable to or greater than that induced by ghrelin is identified as a therapeutic candidate for treating SNpc neurodegeneration.

Test compounds may first be screened for binding to GHSR1a using any methods known in the art for evaluating binding. A non-limiting assay for GHSR binding is a competitive radioligand binding assay (Bednarek et al., 2000, J. Med. Chem. 43:4370-4376; Palucki et al, 2002, Bioorg. Med. Chem. Lett. 11:1955-1957). Those that bind are then tested for the ability to induce at least one of the biological activities discussed above, such as increasing the firing rate of SNpc DA neurons, using methods such as those described in the Examples. Biological activity, such as firing rate, may be assessed in normal SNpc DA neuron cells or may be assessed in SNpc cells from disease models. Action via GHSR can be assessed, for instance, using mice having GHSR knock out with ability to recombine back in, as described in the Examples.

Test compounds for use in the screening methods can be small molecules, nucleic acids, peptides, peptidomimetics and other drugs. The test compounds of the present invention can be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145). Ghrelin mimetics identified by the inventive method may be useful directly in therapeutic applications, and may also serve as lead drugs in the development of further therapeutics.

Examples of methods for the synthesis of molecular libraries may be found in the art, for example, in: DeWitt et al., 1993, Proc. Natl. Acad. Sci. USA 90:6909-6913; Erb et al., 1994, Proc. Natl. Acad. Sci. USA 91:11422-11426; Zuckermann et al., 1994, J. Med. Chem. 37:2678-2685; Cho et al., 1993, Science 261:1303-1305; Carell et al., 1994, Angew. Chem. Int. Ed. Engl. 33:2059-2061; Carell et al., 1994, Angew. Chem. Int. Ed. Engl. 33:2061-2064; and Gallop et al., 1994, J. Med. Chem. 37:1233-1251.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., Proc. Natl. Acad. Sci. USA 89:1865-1869 (1992)), or phage (Scott et al., 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

preparation of a pharmaceutical composition comprising a compound identified by a method of the invention is described elsewhere herein. Compounds which are identified using any of the methods described herein may be formulated and administered to a mammal for treatment of the diseases disclosed herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods used in the following Experimental Examples are now described.

Animals: All procedures described below were approved by the Institutional Animal Care and Use Committee of Yale University. Mice were kept under standard laboratory conditions with free access to food and water. Ghrelin knockout (ghrelin ko; ghrelin^{-/-}) and growth hormone secretagogue receptor knockout (GHSR ko; ghsr^{-/-}) mice were obtained from Regeneron Pharmaceuticals and bred in Yale facilities. These genetic mouse lines have been described previously (Wortley et al., 2004, Proc Natl Acad Sci USA 101: 8227-32). Rats used in the following examples were wild type rats.

Generation of mice expressing ghrelin receptors selectively in catecholaminergic cells: Mice with GHSRs only in catecholaminergic cells were obtained by first crossing mice homozygous for a recombinant “null” GHSR allele (Zigman et al., 2005, J Clin Invest 115: 3564-72) with mice expressing Cre recombinase under the control of a tyrosine hydroxylase promoter (TH-Cre) (Savitt et al., 2005, J Neurosci 25: 6721-8). The recombinant null GHSR allele differs from the wild-type GHSR allele in that the null GHSR allele contains a loxP-flanked transcriptional blocking cassette inserted into an intron located downstream of the transcriptional start site and upstream of the translational start site of the murine GHSR gene. The replacement of two wild-type GHSR alleles with two recombinant GHSR-null alleles results in GHSR-null mice that no longer acutely increase food intake in response to exogenous ghrelin administration and are resistant to the development of diet-induced obesity (Zigman et al., 2005, J Clin Invest 115: 3564-72). The offspring of GHSR-null mice×TH-Cre mice were then bred with mice containing either no, one or two copies of the recombinant null GHSR allele, in order to generate the three desired study animal genotypes: wild-type mice (wt/wt; have two copies of the wild-type GHSR allele and no copies of the TH-Cre transgene), GHSR-null mice (GHSR homo/wt; have two copies of the recombinant null GHSR allele and no copies of the TH-Cre transgene), and “TH-only” mice (GHSR homo/TG; have two copies of the recombinant null GHSR allele and one copy of the TH-Cre transgene).

In the presence of Cre recombinase, the transcriptional blocking cassette and one of the loxP sites is removed from the recombinant null GHSR alleles, thus re-activating the ability to synthesize functional GHSR mRNAs. The specificity of Cre recombinase expression afforded by the promoter elements used in the TH-Cre transgene allows for selective re-activation of GHSR expression within tyrosine hydroxylase-expressing cells that have the latent genetic capacity to express GHSR.

Biotinylated ghrelin binding in the SN: To test whether ghrelin binds to cells in the substantia nigra, 100 μm sections containing these regions were processed for binding studies as described previously (Cowley et al., 2003, Neuron 37: 649-61).

Briefly, saline-perfused rat brains (n=4) were removed, sectioned, and immediately reacted with biotinylated ghrelin (1M; Phoenix Pharmaceuticals, Belmont, Calif.) alone or in combination with an equal amount of unlabeled (cold) ghrelin (1M; Phoenix Pharmaceuticals) for 20 minutes at 4° C. Sections were fixed with 4% paraformaldehyde and reacted with avidin-Texas red and analyzed using a Zeiss microscope equipped with fluorescent filters.

Immunohistochemistry for GHSR and TH: GHSR (anti-GHSR antibody from Phoenix Pharmaceuticals Inc.) and tyrosine hydroxylase (TH) (anti-TH antibody from Chemicon International) were detected in floating sections using standard laboratory procedures described elsewhere (Abizaid et al., 2006, J Clin Invest. 116(12):3229-39. Epub 2006 Oct. 19; Andrews et al., 2005, J Neurosci 25: 184-91). For single label GHSR immunohistochemistry, primary antibody (1:1000) was incubated overnight in a cold room. GHSR was visualized with nickel-diaminobenzidine resulting in a black product. For immunofluorescence, GHSR (1:500) and TH (1:1000) were visualized using appropriate Alexa-fluor secondary antibody conjugates and sections were examined under a Zeiss microscope. Control experiments were conducted by preabsorption of appropriate antisera with their target peptides. GHSR ko mice were also used as a negative control, as no GHSR was observed in these mice.

Patch clamp recordings from VTA dopamine neurons: Brain slices (300 μm) containing the substantia nigra pars compacta (SNpc), were cut on a vibratome from 2-3 week old male and female mice (n=9) and rats (n=5). Briefly, animals were anesthetized with Nembutal (80 milligram/kilogram body weight) and then decapitated. The brains were rapidly removed and immersed in cold (4° C.) oxygenated bath solution (containing (mM): NaCl 150; KCl 2.5; CaCl₂ 2; MgCl₂ 2; Hepes 10; and glucose 10, pH 7.3 with NaOH). After being trimmed to contain only the SNpc, slices were transferred to a recording chamber where they were constantly perfused with bath solution at 2 milliliter/minute. Dopamine neurons in the SNpc were identified by presence of a large Ih current (>100 pA) evoked by hyperpolarizing voltage steps from −50 to −120 mV for 2 seconds. This approach identifies dopaminergic cells with >90% accuracy (Abizaid et al., 2006, J Clin Invest. 116(12):3229-39. Epub 2006 Oct. 19). In brain slices, whole-cell current clamp was used to observe spontaneous action potentials. Slices were maintained at 33° C. and perfused continuously with ASCF (bubbled with 5% CO₂ and 95% O₂) containing (in mM): NaCl, 124; KCl, 3; CaCl₂, 2; MgCl₂, 2; NaH₂PO₄, 1.23; NaHCO₃, 26; glucose, 10, pH 7.4 with NaOH. Ghrelin was applied to the recording chamber via bath application. The pipette solution contained (mM): gluconic acid 140; CaCl₂, MgCl₂ 2; EGTA 1; HEPES 10; Mg-ATP; 4, and Na₂-GTP; 0.5, pH 7.3 with KOH

Real-time PCR: RNA was extracted from the midbrain of 8 ghrelin-treated mice or 8 saline-treated mice using TRIzol® (Invitrogen, Carlsbad, Calif.) according to the manufacturer's instructions. cDNA was synthesized using the First-Strand cDNA synthesis kit (Amersham Biosciences), using a total of 3 μg of total RNA in 15 μl of total volume. Real-time PCR analysis of TH mRNA (SEQ ID NO. 4: 5′ ggaggctttccagcttctg 3′; SEQ ID NO. 5: 5′ gtcagccaacatgggtacg 3′) was performed using iQ SYBR green supermix (Bio-Rad) and 0.5 μm of each primer. Primers for 18S were used as controls. Measurements were performed on an iCycler (Bio-Rad).

Motor behavior: Spontaneous locomotor activity was examined in the ghrelin ko (n=8) and wild type (wt; n=9) at 5 months of age. Activity measures were assessed in a 16 inch square plexiglass arena with photobeams and sensors spaced at intervals sufficient to provide a spatial resolution of animal movement of 1.27 cm in the X-Y dimension (Coulbourn Instruments TruScan, Bilaney Consultants Ltd, Sevenoaks, UK). The arena sensors were computer coupled and sampled every 100 millisecond using Coulbourn Instruments Truscan 99 software. Activity measures were recorded over a 15 minute period and statistical comparisons between ko and wt groups on measures of total distance traveled, average movement velocity and the amount of rest time (time spent not moving) during a 15 minute session. Potential alterations in gross motor coordination and motor learning were assessed using an accelerating Rota Rod. Ghrelin ko and wt mice were placed on a rotating cylinder (AccuScan Instruments, Columbus, Ohio, USA) and the amount of time they were able to walk on the cylinder without falling was recorded for each of six sessions, spaced 5 minutes apart on 2 consecutive days. The rotation speed of the cylinder accelerated from 0 to 40 rpm over a 200 second period and the speed remained constant upon reaching 200 rpm.

Mitochondrial respiration: The ability of ghrelin to promote mitochondrial respiration was examined in mitochondria isolated from midbrain of ghrelin-treated rats (30 nmol) three hours after injection. Rats were used as the mitochondrial yield after isolation is much higher than the yield from mice. Mitochondrial respiration was also examined in the pooled midbrain of UCP2 ko and wt mice (3 pooled brains), 3 hours after ghrelin injection (IP 30 nmol).

Mitochondria were pooled from the SN-VTA (4 animals=1, total n=4) and isolated by differential centrifugation. Because of the size of the tissue dissected, it was impossible to solely isolate only the SNc, and as such the results are reported as SN-VTA. Briefly, the SN-VTA was rapidly dissected and homogenized in isolation buffer (215 mM Mannitol, 75 mM sucrose, 0.1% fatty acid free BSA, 20 mM Hepes, 1 mM EGTA, pH adjusted to 7.2 with KOH). The homogenate was spun at 1300×g for 3 minutes. The supernatant was removed, and the pellet was resuspended with isolation buffer and spun again at 1300×g for 3 minutes. The 2 sets of supernatant from each sample were topped off with isolation buffer and spun at 13000×g for 10 minutes. The supernatant was discarded, and this step was repeated. After this second spin at 13000×g, the supernatant was discarded, and the pellets were resuspended with isolation buffer without EGTA and spun at 10000×g for 10 minutes. The final mitochondrial pellet was resuspended with 50 μl of isolation buffer without EGTA. Protein concentrations were determined with a BCA protein assay kit (Pierce, Rockford, Ill., USA).

Mitochondrial respirations were assessed using a Clark-type oxygen electrode (Hansatech Instruments, Norfolk, England) at 37° C. with pyruvate and malate (5 mM and 2.5 mM) as oxidative substrates in respiration buffer (215 mM Mannitol, 75 mM sucrose, 0.1% fatty acid free BSA, 20 mM Hepes, 2 mM MgC1, 2.5 mM KH₂PO₄, pH adjusted to 7.2 with KOH). Following the addition of ADP and oligomycin, UCP-mediated proton conductance was measured as increased fatty acid-induced respiration (Echtay et al., 2002), which was then compared to state 4 respiration induced by oligomycin, an inhibitor of H⁺ transporting ATP synthase.

UCP2 knockout mice are described in Andrews et al., 2005, J Neurosci 25: 184-91.

Mitochondria number: Animals were perfused and their brains were processed for TH immunolabeling for electron microscopic examination. Ultrathin sections were cut on a Leica ultra microtome, collected on Formvar-coated single-slot grids and analyzed with a Tecnai 12 Biotwin (FEI Company) electron microscope. Mitochondria were counted blindly from randomly selected sections, and Scion Image was used to normalize cytoplasmic area so that mitochondrial number per cell is expressed in square micrometers.

MPTP administration: Mice were injected IP with 30 mg/kg of 1-methyl-4-phenyl-1,2,5,6 tetrahydropyridine (MPTP) in saline as described previously (Andrews et al., 2005, J Neurosci 25: 184-910). Control animals were given saline. Animals were sacrificed and perfused 7 days later and processed for immunohistochemistry or HPLC for dopamine (DA) and metabolites. n=8-10 for ghrelin wt and ko mice; n=6 for GHSR wt/wt, homo/wt and homo/TG.

TH cell quantification: Free floating sections were stained with TH (1:5000, Chemicon International) and visualized with DAB using standard procedures.

Unbiased stereology methods were used to quantify TH immunoreactive cells in the SNpc. Cells were visualized by a Zeiss microscope and relayed via a MicroFibre digital camera (www(dot)optronics(dot)com) to a computer where they were counted using the optical fractionator with the sophisticated Stereolnvestigator software (MicroBrightField, Williston, Vt., USA). Every fourth section was collected through the SNpc, and TH cells were counted in grids randomly positioned by the software in the outlined counting area through all optical planes, thus creating a 3 dimensional counting area. Cells were only counted if they touched the inclusion border or did not touch the exclusion border of the sampling grid.

Striatal DA measurements: At the time of sacrifice (7 days after MPTP treatment), both striata were rapidly dissected on a chilled glass plate and frozen at −70° C. The samples were subsequently thawed in 0.4 ml of chilled 0.1 M perchloric acid and sonicated. Aliquots were taken for protein quantification using a spectrophotometric assay. Other aliquots were centrifuged, and DA levels were measured in supernatants by high pressure liquid chromatography (HPLC) with electrochemical detection. Concentrations of DA and metabolites were expressed as ng/mg protein (mean±SEM).

Statistical Analyses: All data are presented as mean±sem. Statistical differences among groups were determined by unpaired 2-tailed student t-tests or two-way ANOVA followed by bonferroni post hoc test as stated.

The results of the experimental examples are now described.

### Experimental Example 1

### Ghrelin Receptors and Binding in the SNpc

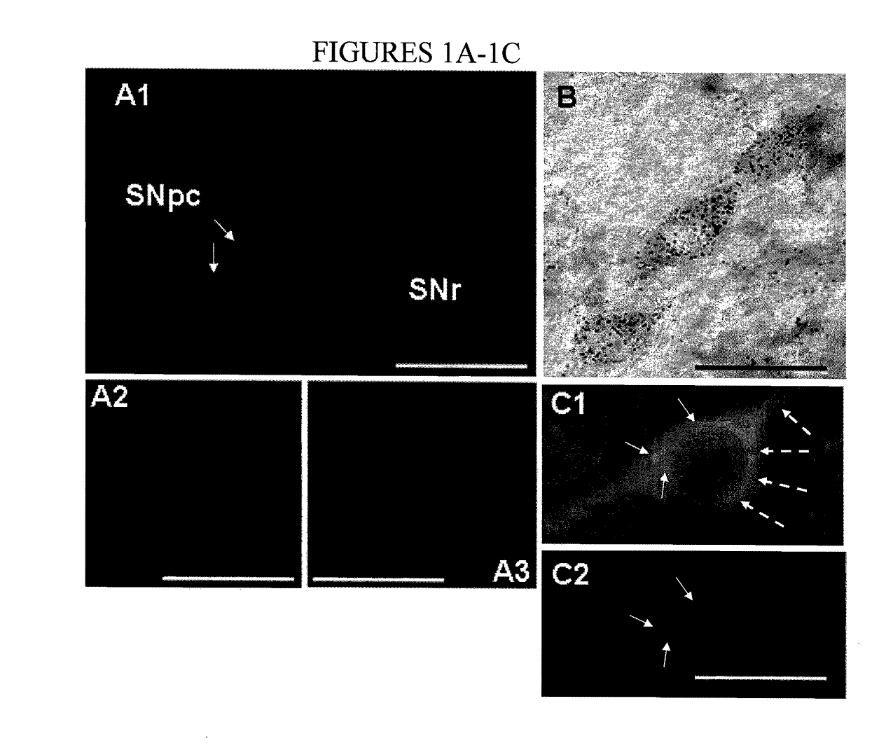

To study ghrelin binding on rat brain slices containing the substantia nigra pars compacta (SNpc), brain sections were reacted with biotinylated ghrelin. Biotinylated ghrelin binding was observed throughout the SNpc (FIGS. 1A1-1A3) with a distribution pattern similar to that in GHSR immunolabelled cells (FIGS. 1B). Ghrelin binding was punctate and associated with neuronal perikarya, similar to GHSR immunostaining, suggesting ghrelin binding to GHSR in the SNpc. No binding was observed when unlabelled ghrelin was added to the incubation solution (FIG. 1A3).

GHSR immunoreactivity was abundant in the SNpc and displayed very similar topography with TH nigral cells. GHSR was characterized by fine punctate staining throughout the rostro-caudal extent of the SNpc (FIG. 1B). Double-labeling showed that greater than 90% present of all TH cells in the SNpc also express GHSR (FIGS. 1C1 and 1 C2), suggesting a functional interaction between ghrelin and nigral dopamine (DA) cells. No staining was observed in sections from GHSR ko (ghsr^{-/-}) mice or in sections where the primary antibody was omitted. These results thus confirm and expand on the anatomical distribution and binding of ghrelin to its cognate receptor GHSR (Guan et al., 1997, Brain Res Mol Brain Res 48: 23-9).

### Experimental Example 2

Ghrelin Increases the Firing Rate of SNpc DA Neurons

The anatomical observations suggest that ghrelin may have a functional role on SNpc DA neurons. To address this question, an experiment using whole-cell patch clamp electrophysiology was performed to investigate whether ghrelin directly activates SNpc DA neurons. DA neurons (n=11) in the SNpc were identified based on their characteristic Ih currents (Johnson et al., 1992, J Neurosci 12: 483-8). Spontaneous action potentials were recorded under current clamp for at least 10 minutes of stable recording. Ghrelin (1-3 μmol) was applied via bath application.

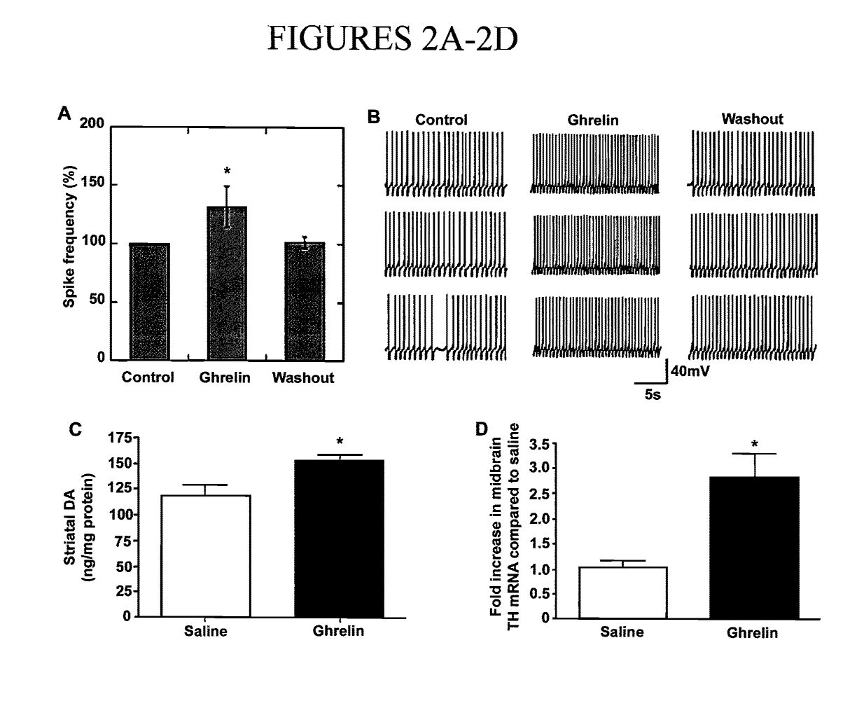

Ghrelin significantly increased action potential frequency above baseline (control 100%, ghrelin 131.9%±17.5%, washout 101.9%±4.6%, p<0.05; FIGS. 2A and 2B). Washout reduced ghrelin-elevated firing back to control levels. Thus, these data provide direct evidence that ghrelin promotes action potential firing in SNpc DA neurons. Furthermore, 10 of 11 identified DA neurons responded to ghrelin, supporting the anatomical evidence that more than 90% of SNpc dopamine express GHSR.

### Experimental Example 3

### Ghrelin Increases Striatal DA Levels

To further examine the effect of ghrelin on the nigrostriatal system, striatal DA concentration after ghrelin injection in wild-type mice was measured using HPLC.

Ghrelin produced a robust and reproducible increase in DA concentration in both the dorsal (n=7, saline 119.4±9.6 vs ghrelin 153±4.6 ng/mg protein, p<0.05; FIG. 2C) and ventral striata (n=7, saline 74.6±126.5±7.26, p<0.05). Ghrelin had no effect on DA concentration in ghsr^{-/-} mice. These results are consistent with a recent study (Abizaid et al., 2006, J Clin Invest 116(12):3229-39. Epub 2006 Oct. 19). The results are also consistent with studies using in vivo microdialysis, in which ghrelin increases extracellular DA concentration in the ventral striatum (Jerlhag et al., 2006, Addict Biol 11: 45-54; Jerlhag et al., 2007, Addict Biol 12: 6-16).

### Experimental Example 4

### Ghrelin Increases TH mRNA Levels in the SN

Tyrosine hydroxylase (TH) is the rate limiting enzyme of dopamine biosynthesis. An experiment was performed to examine whether ghrelin alters TH mRNA expression in the midbrain.

Three hours after ghrelin injection (10 nmols) in wild-type mice, TH mRNA expression was significantly increased in the midbrain compared to saline-injected controls (n=10 per group, saline 1.03±0.15 vs ghrelin 2.86±0.46, p=0.0013; FIG. 2D).

### Experimental Example 5

Ghrelin^{-/-} Mice Display Altered Locomotor Behavior

Parkinson's Disease (PD) is characterized by motor dysfunction. Given the results showing ghrelin's effects on the nigrostriatal DA system, an experiment was performed to investigate whether the absence of ghrelin would have observable effects on motor functions.

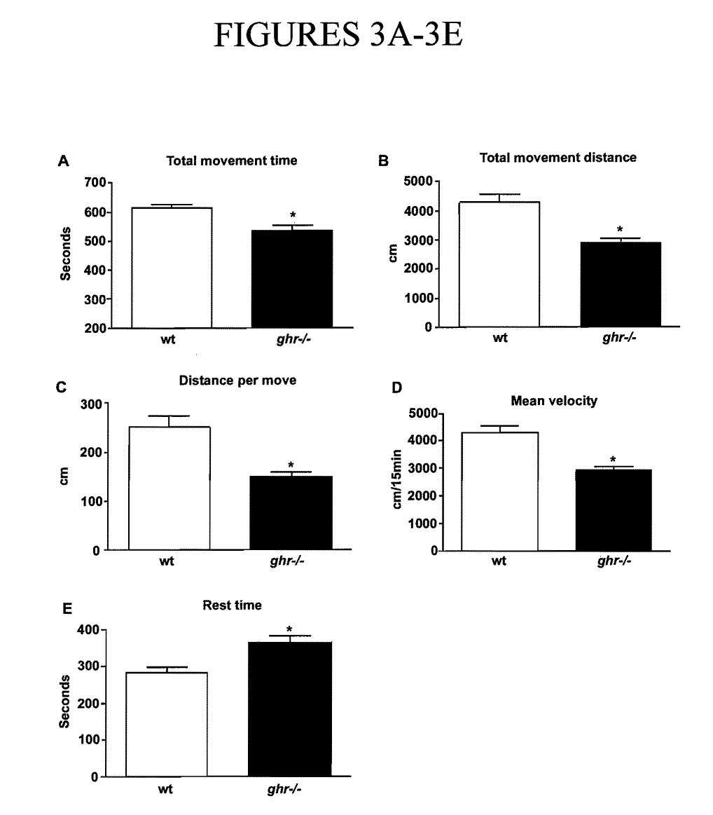

The comparison of 15 minute open field activity between ghrelin^{+/+} (n=9) and ghrelin^{-/-} (n=8) mice revealed that ghrelin^{-/-} mice have a 13% decrease in total movement time (wt 613.3±12.4 vs ko 535.3±17.0 seconds, p=0.002; FIG. 3A), a 32% decrease in total movement distance (wt 4271±264 vs ko 2892±121 cm, p=0.0004; FIG. 3B), a 40% decrease in distance per move (wt 250.1±22.8 vs ko 149.9 cm, p=0.001; FIG. 3C), a 32% decrease in mean velocity (wt 4293.5±264.4 vs ko 2914.5±120.5 cm/15 minutes, p=0.0004; FIG. 3D), and a 29% increase in rest time (wt 282.7±14.3 vs ko 364.8±17 seconds, p=0.003; FIG. 3E). These data are consistent with many recent studies showing that ghrelin affects locomotor activity (Jerlhag et al., 2006, Addict Biol 11: 45-54; Jerlhag et al., 2007, Addict Biol 12: 6-16; Matsuda et al., 2006, Peptides 27: 1335-40; Wellman et al., 2005, Regul Pept 125: 151-4).

### Experimental Example 6

### Ghrelin Influences Midbrain Mitochondrial Efficiency

Recent studies showed that ghrelin can affect mitochondrial metabolism by reducing reactive oxygen species, stabilizing the mitochondrial membrane potential, increasing the Bcl-2/Bax ratio, preventing caspase 3 activation (Chung et al., 2007, Endocrinology 148, 148-59) and regulating mitochondrial gene expression (Barazzoni et al., 2005, Am J Physiol Endocrinol Metab 288: E228-35). To determine whether ghrelin influences mitochondrial metabolism in the SNpc, mitochondrial respiration was measured in isolated synaptosomes from the midbrain of ghrelin-treated wild-type rats.

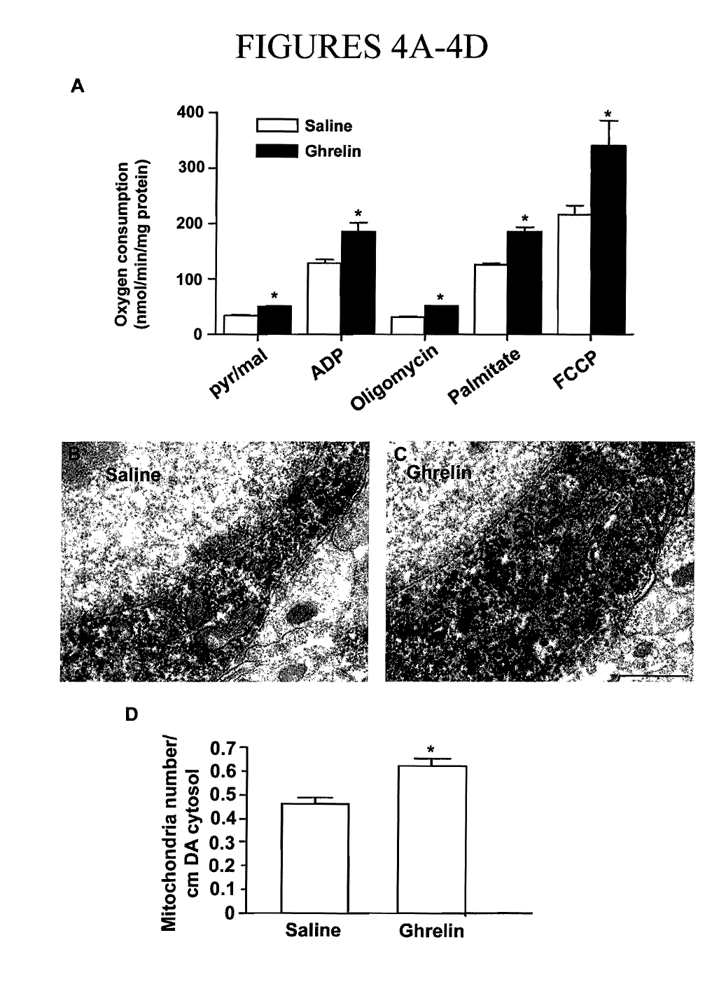

Ghrelin was administered intraperitoneally (30 nmol) and food was removed for the three hours injection period prior to sacrifice as ingested calorie load and the postprandial increase in glucose and insulin are known to reduce ghrelin effectiveness (Callahan et al., 2004, J Clin Endocrinol Metab 89: 1319-24; Cummings et al., 2006, Physiol Behav 89: 71-84). The addition of primary energy substrates pyruvate and malate led to greater respiration in response to ghrelin (saline 33.5±2.1 vs ghrelin 49.5±1.9, p<0.001; FIG. 4A) suggesting greater electron supply via NADH. Ghrelin also induced a greater rate of respiration after the addition of ADP (saline 128.3±7.5 vs ghrelin 185.2±17.7, p<0.05; FIG. 4A) indicating greater ADP phosphorylation via the ATP synthase. Subsequent blocking of the ATP synthase with oligomycin produced greater basal uncoupling after ghrelin (saline 31.4±1.4 vs ghrelin 50.4±1.5, p<0.0001; FIG. 4A). Palmitate (free fatty-acid) induced respiration was also elevated in ghrelin-treated rats (saline 125.0±3.8 vs ghrelin 185.3±9.3, p<0.001; FIG. 4A). Finally, ghrelin increased FCCP-driven respiration, an index of total uncoupling capacity of the inner mitochondrial membrane (FIG. 4A).

### Experimental Example 7

### Ghrelin Induces Increased Mitochondria Number in SNpc DA Perikarya

An increase in mitochondrial respiration is associated with mitochondrial biogenesis (Diano et al., 2003, Endocrinology 144: 5014-21; Wu et al., 1999, Cell 98: 115-24), and an increase in midbrain mitochondrial respiration increases mitochondrial number and decreases vulnerability of midbrain DA neurons in mouse and non-human primate models of Parkinson's disease (Andrews et al., 2005, J Neurosci 25: 184-91; Conti et al., 2005, J Neurochem 93: 493-501; Horvath et al., 2003, Endocrinology 144: 2757-60).

Given that ghrelin induces mitochondrial respiration as described above, an experiment was performed to test whether ghrelin could also alter mitochondrial number in the SNpc DA perikarya. Three hours after ghrelin injection to mice, an increased mitochondrial number in DA perikarya of the SNpc was observed (saline 0.46±0.03 vs ghrelin 0.62±0.03 μm²; FIGS. 4B-4D).

### Experimental Example 8

### Ghrelin and Toxic Insult

After establishing that ghrelin targets the SNpc and produces a functional effect, an experiment was designed to determine if ghrelin plays a role in recovery after a challenge with the mitochondrial toxin, MPTP.

Ghrelin ko and wt mice were weighed 1 day after MPTP or saline injection and again at sacrifice 7 days later. Wt and ko mice treated with saline gained a similar amount of weight over the 7 days (wt saline 1.39±0.19 g vs ko saline 1.59±0.49 g). MPTP acutely reduces body weight after 1 day, which then rebounds after 7 days, thus wt mice treated with MPTP gained 2.16±0.10 g over the experimental period. However, ghrelin ko mice treated with MPTP did not produce the anticipated body weight gain observed in the wt types (wt MPTP 2.17±0.10 vs ko MPTP 1.37±0.15, p<0.05). As ghrelin's most well described role involves increasing food intake and adiposity through a hypothalamic action, the lack of body weight gain in ghrelin ko mice after MPTP suggests that ghrelin plays an important role in reestablishing body weight after a toxic insult. Moreover, the lack of compensation may exacerbate the toxic affect of MPTP.

### Experimental Example 9

### Ghrelin Protects Against MPTP-Induced SNpc DA Cell Loss

The observation that ghrelin promotes SNpc DA neuronal function, mitochondrial respiration and number raises the possibility that ghrelin may protect nigrostriatal dopaminergic system during increased cellular stress. In order to test whether ghrelin could restrict DA cell loss in the SNpc, wt mice were given ghrelin 7 days prior to, and 7 days after, MPTP intoxication. In brief, mice were treated with daily IP injections of exogenous ghrelin (10 nmols) or saline for 14 days. Mice were injected immediately before the dark phase, and food was removed overnight since calorie intake and postprandial increases in glucose and insulin can suppress the action of ghrelin (Cummings et al., 2006, Physiol Behav 89: 71-84). Both ghrelin and saline treated mice were given 3 grams of standard chow the following morning at 9 am. On the seventh day, MPTP, a mitochondrial toxin that models PD by ablating nigral DA neurons, was administered IP (30 mg/kg) to both the saline and ghrelin-treated mice. After the subsequent 7 days of treatment (saline or ghrelin), TH immunoreactive neurons were counted blind by two independent researchers using the non-biased optical fractionator on the stereology program, Stereoinvestigator.

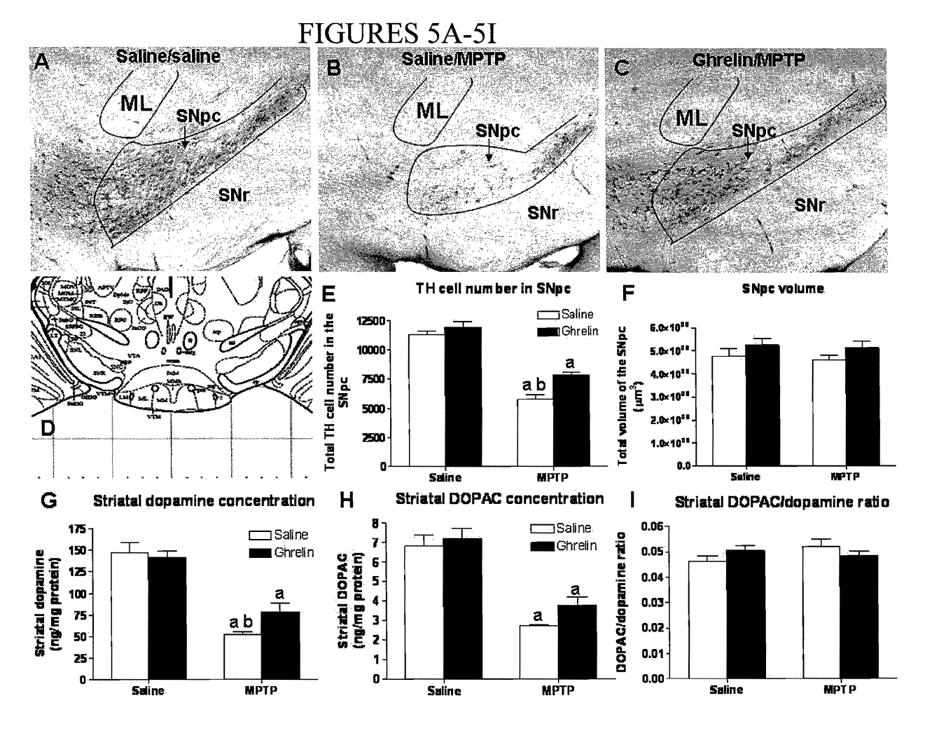

FIGS. 5A-5C are representative images of striata immunostained for TH from control mice (14 days saline; saline injection), MPTP-treated mice (14 days saline; MPTP injection) and ghrelin and MPTP-treated mice (14 days ghrelin; MPTP injection). FIG. 5D is an image from the mice brain atlas. Comparison of FIGS. 5B and 5C reveals qualitatively a difference in the extent of striatal dopamine loss due to MPTP. Specifically, administration of ghrelin noticeably reduces the extent of MPTP-induced dopamine cell loss.

Quantitatively, there was no difference in estimated total number of TH cells in the entire SNpc in mice treated with ghrelin and saline (at day 7; designated ghr/sal) or saline and saline (at day 7; designated sal/sal) (total TH neurons=sal/sal 11283±342 vs ghr/sal 11616±318, n=6; FIG. 5E, left bars). Similarly, the SNpc volume was not significantly different (FIG. 5F, left bars). Mice treated with saline followed by MPTP (on day 7; designated sal/MPTP) and mice treated with ghrelin followed by MPTP (on day 7; designated ghr/MPTP) both displayed significantly TH cell loss in the SNpc (FIG. 5E, right bars), although SNpc volume was not significantly different (FIG. 5F, right bars). However, 14 day ghrelin treatment significantly attenuated TH cell loss in the SNpc in response to MPTP compared to sal/MPTP controls (sal/MPTP 5750±432 vs ghr/MPTP 7836±285, n=6, p<0.05). Indeed ghrelin treatment restricted dopamine cell loss to 33% of control mice, whereas saline treatment resulted in 49% dopamine cell loss compared to controls after MPTP lesion. These data indicate that ghrelin promoted dopamine SNpc cell survivability after MPTP intoxication.

Striatal dopamine concentrations were not statistically different in sal/sal or ghr/saline mice (sal/sal 147.4±10.2 vs ghr/sal 141.8±7.5 ng/mg protein, n=6; FIG. 5G, left bars). In parallel with dopamine cell loss in the SNpc, MPTP significantly reduced striatal dopamine in both saline and ghrelin treated mice; however ghrelin significantly attenuated the MPTP-induced loss of dopamine (sal/MPTP 51.4±2.7 vs ghr/MPTP 78.4±8.8 ng/mg protein, n=6, p<0.05; FIG. 5G, right bars). The loss of dopamine in sal/MPTP mice was 65% lower than controls whereas ghr/MPTP only showed a 45% loss of striatal dopamine. The level of DOPAC, a metabolite of dopamine, generally paralleled the level of dopamine (FIGS. 5H and 5I). DOPAC and DOPAC/dopamine are commonly reported indices of dopamine metabolism at the nerve terminal.

These experiments successfully demonstrate that ghrelin can restrict dopamine cell loss in a model of Parkinson's Disease. Consequently, ghrelin represents a novel therapeutic for the treatment of PD patients.

### Experimental Example 10

### Ghrelin and UCP2-Dependent Respiration

Previous studies have shown that uncoupling protein 2 (UCP2) restricts MPTP-induced dopamine loss (Andrews et al., 2005, J Neurosci 25:184-191; Conti et al., 2005, J Neurochem 93:493-501) and that ghrelin increases hypothalamic UCP2-dependent respiration and gene expression (Andrews 2007, manuscript submitted). Having determined that ghrelin protects against MPTP-induced striatal dopamine loss and nigral dopamine cell loss, an experiment was designed to test whether ghrelin promotes nigral dopamine cell function by activating UCP2-dependent respiration.

It was determined that GHSRs are expressed on UCP2 neurons in the SNpc. Given that GHSRs and UCP2 are both colocalized with TH in the SNpc (Conti et al., 2005, J Neurochem 93:493-501), it is concluded that TH neurons in the SNpc contain both GHSR and UCP2. To test whether UCP2 is important in ghrelin-induced midbrain respiration, mitochondria were isolated from the midbrain of pooled UCP2 wt or ko mice treated with ghrelin or saline, and uncoupled respiration was measured.

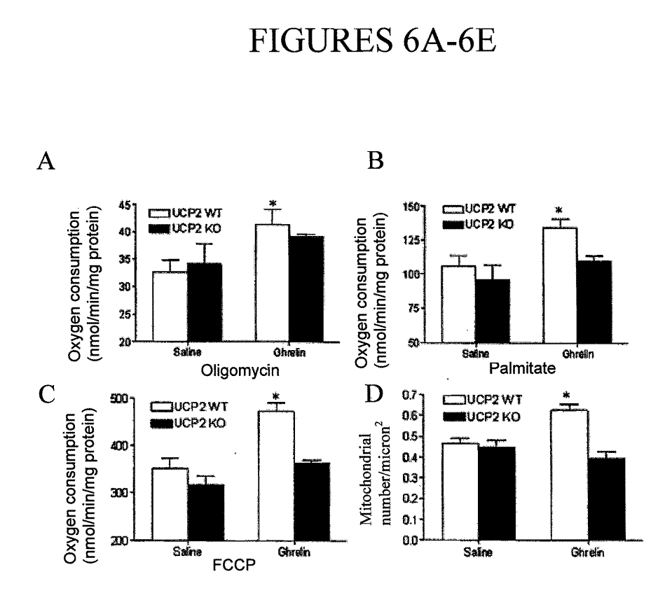

Ghrelin-treated UCP2 wt mice exhibited significantly greater basal uncoupled respiration after oligomycin-blocked electron transport through the ATP synthase (wt saline 32.7±1.6 vs wt ghrelin 41.5±1.9, p<0.05), whereas ghrelin did not increase respiration after oligomycin in UCP2 ko mice (ko saline 34.2±2.6 vs ko ghrelin 39.2±0.4; FIG. 6A). Activation of UCP2 with the fatty acid palmitate (Echtay et al., 2002, Nature 415:96-9) resulted in a significant increase in mitochondrial respiration in ghrelin treated UCP2 wt but not ko mice (wt saline 106.3±5.3 vs wt ghrelin 134.5±4.5, p<0.05; ko saline 96.0±7.5 vs ko ghrelin 109.7±2.9 (FIG. 6B). Finally, FCCP-driven respiration, which measures the total uncoupling capacity, was also increased in ghrelin-treated UCP2 wt but not ko mice (wt saline 352±14.1 vs wt ghrelin 472±12.7, p<0.05; ko saline 3 17.0±12.7 vs ko MPTP 363.5±3.8) (FIG. 6C).

UCPs are heavily regulated in vivo by fatty acids (Echtay et al., 2002, Nature 415:96-99; Zackova et al., 2003, J Biol Chem 278:20761-20769). Without wishing to be limited by theory, it is contemplated that the mechanism responsible for ghrelin-induced uncoupled respiration may be related to its unique biochemical feature of a fatty acid (n-octanolyl) side chain at serine 3 that confers it its bioactivity (Hosoda et al., 2000, Biochem Biophys Res Commun 279:909-913). Octanoate is known to increase mitochondrial proton leak and uncoupled respiration in hepatocytes (Leverve et al., 1998, Mol Cell Biochem 184:53-65; Nobes et al., 1990, J Biol Chem 265:12910-12915), which also supports this mechanism.

UCP2 is important in promoting mitochondrial biogenesis in response to increased uncoupled respiration in many tissues including the brain (Diano et al., 2003, Endocrinol 144:5014-5021), muscle (Cha et al., 2006, PNAS 103:15410-15415; Wu et al., 1999, Cell 98:115-124) and brown adipose tissue (Rossmeisl et al., 2002, Eur J Biochem 269:19-28). Furthermore, ghrelin levels in the circulation are elevated during fasting, and fasting is associated with UCP2-dependent increase of mitochondrial number in NPY/AgRP neurons (Coppola et al., 2007, Cell Metab 5:21-33) in order to drive food intake.

Therefore, an experiment was performed to test whether ghrelin increases mitochondrial number in the SNpc dopamine neurons. Two hours after ghrelin injection, UCP2 wt mice exhibited increased mitochondrial number (saline 0.46±0.03 vs ghrelin 0.62±0.03 μm²), however ghrelin had no effect on mitochondrial number in UCP2 ko mice (saline 0.45±0.03 vs ghrelin 0.39±0.03 μm²; FIG. 6D). These data suggest that ghrelin's neuroprotective effect involves UCP2-dependent maintenance of mitochondrial number after MPTP intoxication. In line with this, UCP2 ko mice show reduced mitochondrial number in the SNpc dopamine neurons, which predisposes them to environmental toxins (Andrews et al., 2005, J Neurosci 25:184-191). Furthermore, mitochondrial dysfunction lies at the very heart of Parkinson's disease (Lin et al., 2006, Nature 443:787-795).

### Experimental Example 11

### Ghrelin Receptor Signaling in Parkinson's Disease

To further examine the importance of ghrelin and GHSR receptor signaling in PD, the effect of MPTP was analyzed in transgenic animals in which either ghrelin or the ghrelin receptor, GHSR, was ablated. Mice in which GHSR was selectively restored in catecholaminergic cells of ghsr^{-/-} animals were also studied (see Experimental Example 12).

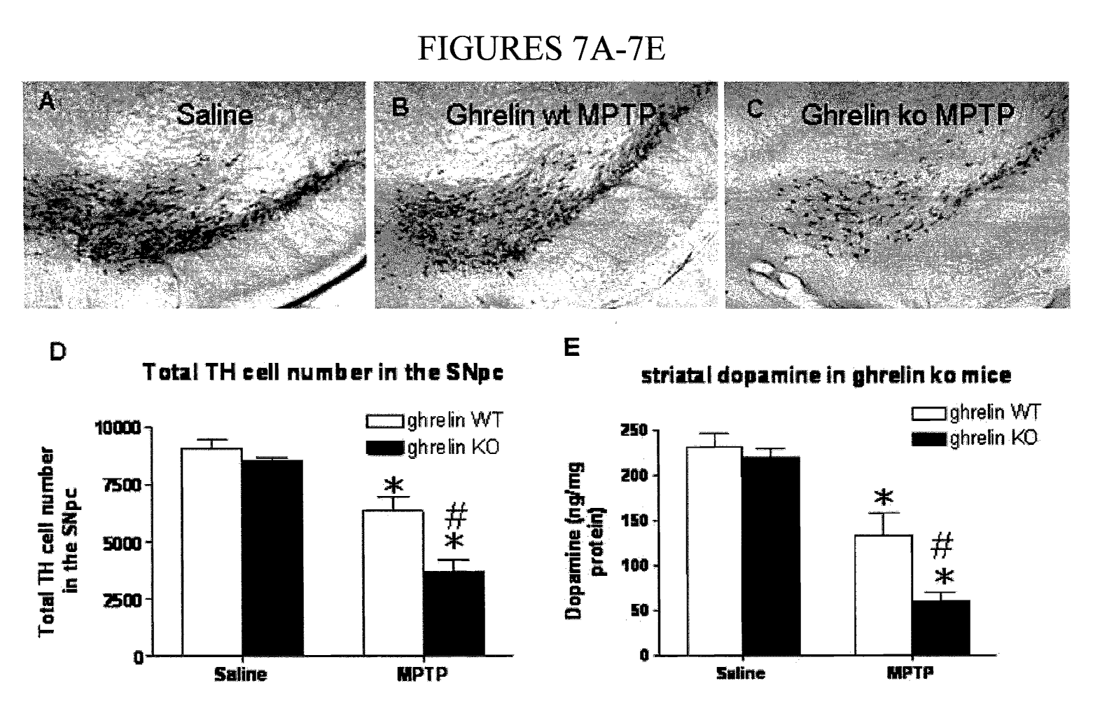

As the images in FIGS. 7A-7C depict, a difference in DA neurons was observed. Specifically, there was a significant loss of DA neurons in the ghrelin ko mice treated with MPTP, compared to ghrelin wt MPTP-treated mice. After saline injection, there was no observable difference in TH-immunoreactive cells in the SNpc of ghrelin^{-/-} mice compared to wild type littermates (wt 9105±342 vs ko 8580±78, P>0.05; FIG. 7D). MPTP produced a significant loss of DA neurons in both ghrelin^{+/+} and ghrelin^{-/-} mice compared to saline controls, however the loss of DA neurons in ghrelin^{-/-} mice was significantly increased, such that ghrelin^{-/-} mice lost almost 50% more DA neurons compared to wt mice (ghrelin^{+/+} MPTP 6688±610 vs ghrelin^{-/-} MPTP 3691±550, p<0.001; FIG. 7D).

To determine whether this loss of TH-immunolabelled neurons correlated with altered dopamine (DA) levels, DA content in the dorsal striatum was measured. There was no statistical difference in DA content between ghrelin and ghrelin^{-/-} mice injected with saline (ghrelin^{+/+} saline 231±21 vs ghrelin^{-/-} saline 219±13 ng/mg protein; FIG. 7E). However, ghrelin^{-/-} mice challenged with MPTP exhibited a significantly greater reduction in striatal DA content compared to ghrelin mice treated with MPTP (ghrelin^{+/+}MPTP 133±32 vs ghrelin^{-/-} MPTP 60±19 ng/ml protein, p<0.001; FIG. 7E). GHSRs are not found in the striatum (Zigman et al., 2006, J Comp Neurol 494: 528-48). Without being bound by theory, it is believed that the decrease in DA content is most likely directly related to the reduction of DA cell number in the SNpc, rather than a trophic role of ghrelin at the level of the nerve terminals.

### Experimental Example 12

### GHSR Receptor Signaling in Parkinson's DISEASE

After determining that ghrelin^{-/-} mice are more susceptible to MPTP, an experiment was performed to determine whether GHSR mediates this effect of ghrelin.

The experimental approach utilized GHSR null mice (designated GHSR homo/wt), which were generated by inserting a loxP-flanked transcription blocking cassette into the endogenous GHSR allele (Zigman et al., 2005, J Clin Invest 115: 3564-72). These GHSR null mice are unique in that when exposed to Cre recombinase, the expression of GHSR can be re-established. GHSR null mice were used to generate mice that express GHSR selectively in TH neurons (designated GHSR homo/TG), including neurons in the SNpc and VTA; these mice were generated by crossing GHSR null mice with TH cre mice. The experiment also used control mice (designated wt/wt).

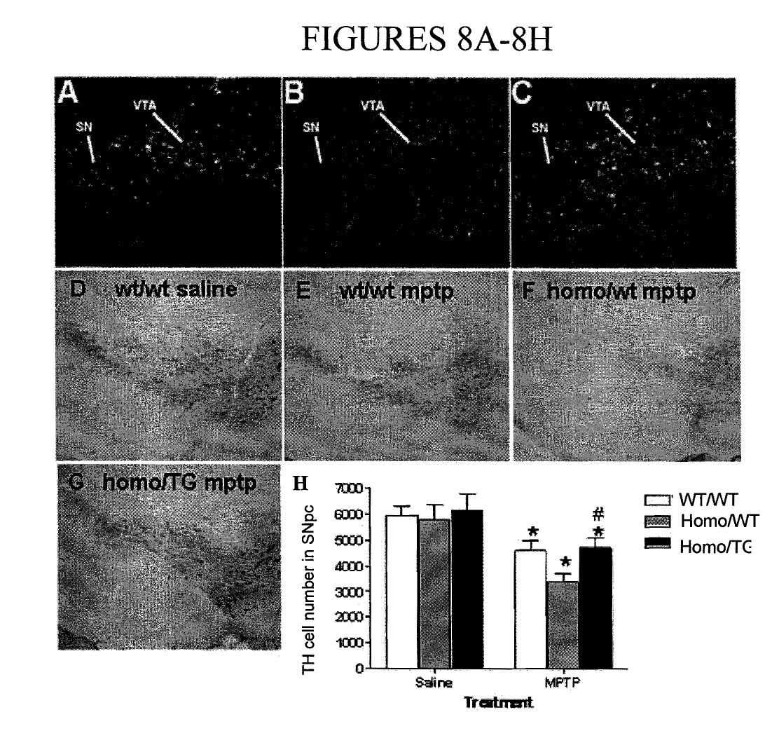

To validate the re-activation of GHSR expression selectively within TH cells of “TH-only” mice, brains were examined for the presence of GHSR mRNA by in situ hybridization histochemistry (ISHH), using a mouse GHSR-specific riboprobe, as previously described (Zigman et al., 2006, J Comp Neurol 494: 528-48). GHSR transcripts were consistently visualized by ISHH within the substantia nigra and ventral tegmental area (FIGS. 8A-8C), within certain hypothalamic nuclei (the arcuate nucleus, the anteroventral periventricular nucleus, the dorsomedial nucleus, and the capsule of the ventromedial nucleus), and occasionally within scattered cells of the nucleus of the solitary tract. The pattern of GHSR expression observed within the “TH-only” mice is similar to the GHSR-TH co-expression pattern that is observed in the wild-type mouse brain and involves a few more sites than what has previously been described in the rat brain (Zigman et al., 2006, J Comp Neurol 494: 528-48). Similar to what was previously described for GHSR null mice (Zigman et al., 2005, J Clin Invest 115: 3564-72), strong binding of the GHSR riboprobe was also observed in the Edinger-Westphal nucleus of “TH-only” mice, which is neither an area of TH expression nor TH-Cre activity.

To perform the experiment of this example, mice were injected with MPTP or saline, and TH neurons in the SNpc were counted using the optical fractionator (FIGS. 8D-8G). While MPTP significantly reduced TH neurons in SNpc of wt/wt mice (wt/wt saline 11902±713 vs wt/wt MPTP 9171±765, p<0.05; FIG. 8H), it produced a significantly greater loss of TH neurons in the SNpc in GHSR homo/wt mice (homo/wt saline 11590±1133 vs 6582±636, p<0.05; wt/wt MPTP 9171±765 vs homo/wt MPTP 6582±636, p<0.05; FIG. 8H). However, mice with GHSR reactivation in TH neurons exhibited restricted MPTP TH cell loss similar to that seen in wt/wt MPTP-treated mice (saline homo/TG 12293±1123 vs MPTP homo/TG 9401±743; wt/wt MPTP 9171±765 vs homo/TG MPTP 9401±743, ns; FIG. 8H). These data demonstrate that ghrelin signaling directly in TH neurons, via GHSR, is critical to maintain ghrelin's neuroprotective properties.

Thus, the results of the Experimental Examples show that ghrelin promotes the electrical activity and dopamine output of the nigrostriatal DA system. Furthermore, the results show that ghrelin has neuroprotective effects in an art-recognized model of PD. Accordingly, exogenously-administered ghrelin and ghrelin mimetics offer a treatment for diseases and disorders featuring degeneration of SNpc dopamine neurons, such as in Parkinson's disease.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating neurodegeneration of a substantia nigra pars compacta (SNpc) dopamine neuron in a mammal, said method comprising administering a therapeutically effective amount of ghrelin or a ghrelin mimetic to a mammal diagnosed with SNpc neurodegeneration, wherein said ghrelin or ghrelin mimetic induces increased dopamine neuron function in said mammal, thereby treating SNpc neurodegeneration.

2. The method of claim 1, wherein said mammal is a human and said human has Parkinson's disease.

3. The method of claim 1, wherein increased dopamine neuron function comprises at least one of: increased firing rate of SNpc DA neurons, increased dopamine concentration in dorsal striatum, increased tyrosine hydroxylase mRNA, increased mitochondrial respiration and increased mitochondrial proliferation.

4. The method of claim 1, wherein ghrelin is administered.

5. The method of claim 1, wherein said ghrelin mimetic is selected from the group consisting of: LY444711, MK-677, L-692,429, CP-424,391, NNC 26-0703, Growth hormone (GH) releasing hexapeptide (GHRP)-6, EP 1572 and Ape-Ser(Octyl)-Phe-Leu-aminoethylamide.

6. The method of claim 1, wherein said administration is selected from the group consisting of parenteral, oral, intranasal and recombinant.

7. The method of claim 6, wherein said administration is recombinant.

8. The method of claim 6, wherein said administration is parenteral.

9. A method of activating a dopamine neuron of the substantia nigra pars compacta (SNpc), said method comprising administering a therapeutically effective amount of ghrelin or a ghrelin mimetic to a SNpc dopamine (DA) neuron, wherein said ghrelin or ghrelin mimetic increases firing rate of said SNpc DA neuron.

10. The method of 9, wherein said SNpc DA neuron is in vitro.

11. The method of 9, wherein said SNpc DA neuron is in vivo in a mammal.

12. The method of claim 9, wherein ghrelin is administered.

13. The method of claim 9, wherein said ghrelin mimetic is selected from the group consisting of: LY444711, MK-677, L-692,429, CP-424,391, NNC 26-0703, Growth hormone (GH) releasing hexapeptide (GHRP)-6, EP 1572 and Ape-Ser(Octyl)-Phe-Leu-aminoethylamide.

14. The method of claim 11, wherein said administration is selected from the group consisting of parenteral, oral, intranasal and recombinant.

15. The method of claim 14, wherein said administration is recombinant.

16. The method of claim 14, wherein said administration is parenteral.

17. A method of reducing weight loss associated with Parkinson's disease in a human having Parkinson's disease, said method comprising administering a therapeutically effective amount of ghrelin or a ghrelin mimetic to said human having Parkinson's disease, wherein said ghrelin or ghrelin mimetic increases appetite in said human.

18. The method of claim 17, wherein ghrelin is administered.

19. The method of claim 17, wherein said ghrelin mimetic is selected from the group consisting of: LY444711, MK-677, L-692,429, CP-424,391, NNC 26-0703, Growth hormone (GH) releasing hexapeptide (GHRP)-6, EP 1572 and Ape-Ser(Octyl)-Phe-Leu-aminoethylamide.

20. The method of claim 17, wherein said administration is selected from the group consisting of parenteral, oral, intranasal and recombinant.

21. The method of claim 20, wherein said administration is recombinant.

22. The method of claim 20, wherein said administration is parenteral.

23. A method of assessing if a mammal is at risk of developing SNpc neurodegeneration, said method comprising assessing endogenous production and/or secretion of ghrelin in said mammal, wherein if the production and/or secretion of ghrelin is reduced compared to a reference level, said mammal is at risk of developing SNpc neurodegeneration.

24. The method of claim 23, wherein said mammal is a human.

25. The method of claim 23, wherein assessing endogenous production and/or secretion of ghrelin comprises one of an immunoassay or a gene expression assay.


SEQUENCE LISTING (5 sequences)

SEQ ID NO: 1 - protein, 28 aa, Homo sapiens
  MISC_FEATURE (3)..(3): acylation site
GSSFLSPEHQRVQQRKESKKPPAKLQPR

SEQ ID NO: 2 - DNA, 84 bp, Homo sapiens
ggctccagcttcctgagccctgaacaccagagagtccagcagagaaaggagtcgaagaagccaccagccaagctgcagccccga

SEQ ID NO: 3 - protein, 4 aa, Homo sapiens
  MISC_FEATURE (1)..(4): Residues 1-4 of human mature ghrelin
GSSF

SEQ ID NO: 4 - DNA, 19 bp, Artificial
  synthetic oligonucleotide
ggaggctttccagcttctg

SEQ ID NO: 5 - DNA, 19 bp, Artificial
  synthetic oligonucleotide
gtcagccaacatgggtacg